SEQUENCE LISTING (3 sequences)

SEQ ID NO: 1 - DNA, 20 bp, Artificial
  primer
gacgggtgagtaacacgtgg

SEQ ID NO: 2 - DNA, 15 bp, Artificial
  primer
acgggcggtgtgtac

SEQ ID NO: 3 - DNA, 1308 bp, Weissella cibaria BLS13
gacgggtgagtaacacgtgggaaacctacctcttagcaggggataacatttggaaacagatgctaataccgtataacaatagcaaccgcatggttgctacttaaaagatggttctgctatcactaagagatggtcccgcggtgcattagttagttggtgaggtaatggctcaccaagacgatgatgcatagccgagttgagagactgatcggccacaatgggactgagacacggcccatactcctacgggaggcagcagtagggaatcttccacaatgggcgaaagcctgatggagcaacgccgcgtgtgtgatgaagggtttcggctcgtaaaacactgttgtaagagaagaatgacattgagagtaactgttcaatgtgtgacggtatcttaccagaaaggaacggctaaatacgtgccagcagccgcggtaatacgtatgttccaagcgttatccggatttattgggcgtaaagcgagcgcagacggttatttaagtctgaagtgaaagccctcagctcaactgaggaattgctttggaaactggatgacttgagtgcagtagaggaaagtggaactccatgtgtagcggtgaaatgcgtagatatatggaagaacaccagtggcgaaggcggctttctggactgtaactgacgttgaggctcgaaagtgtgggtagcaaacaggattagataccctggtagtccacaccgtaaacgatgagtgctaggtgtttgagggtttccgcccttaagtgccgcagctaacgcattaagcactccgcctggggagtacgaccgcaaggttgaaactcaaaggaattgacggggacccgcacaagcggtggagcatgtggtttaattcgaagcaacgcgaagaaccttaccaggtcttgacatcccttgacaactccagaaatggagcgttcccttcggggacaaggtgacaggtggtgcatggttgtcgtcagctcgtgtcgtgagatgttgggttaagtcccgcaacgagcgcaacccttattactagttgccagcattcagttgggcactctagtgagactgccggtgacaaaccggaggaaggtggggatgacgtcaaatcatcatgccccttatgacctgggctacacacgtgctacaatggcgtatacaacgagttgccaacccgcgagggtgagctaatctcttaaagtacgtctcagttcggattgtaggctgcaactcgcctacatgaagtcggaatcgctagtaatcgcggatcagcacgccgcggtgaatacgttcccgggtcttgtacacaccgcccgt

(19) United States
(12) Patent Application Publication
(10) Pub. No.: US 2012/0156172 A1
(43) Pub. Date: Jun. 21, 2012

(54) NEW LACTIC ACID BACTERIA HAVING ITS INHIBITORY EFFECT ON AVIAN INFLUENZA VIRUS INFECTION AND COMPOSITION CONTAINING THE SAME

(75) Inventors: Jong-Bok Rho, Seoul (KR); Haryoung Poo, Daejeon (KR); Young-Ki Choi, Cheongju-si (KR); Chul Joong Kim, Daejeon (KR); Moon-Hee Sung, Daejeon (KR)

(73) Assignees: BIOLEADERS CORPORATION, Daejeon (KR); KOREA RESEARCH INSTITUTE OF BIOSCIENCE AND BIOTECHNOLOGY, Daejeon (KR); THE INDUSTRY AND ACADEMIC COOPERATION IN CHUNGNAM NATIONAL UNIVERSITY, Daejeon (KR); KOOKMIN UNIVERSITY INDUSTRY-ACADEMIC COOPERATION FOUNDATION, Seoul (KR); CHUNGBUK NATIONAL UNIVERSITY INDUSTRY ACADEMIC COOPERATION, Cheongju-si ,Chungcheongbuk-do (KR)

(21) Appl. No.: 13/378,761

(22) PCT Filed: Jun. 17, 2010
(86) PCT No.: PCT/KR2010/003895

§ 371 Date: Mar. 6, 2012

(30) Foreign Application Priority Data
Jun. 17, 2009 (KR) 10-2009-0053845

(51) Int. Cl. A61K 35/74; A61P 31/16; C12N 1/20

(52) U.S. Cl. 424/93.4; 435/252.1

(57) ABSTRACT

The present invention relates to a new microorganism Weissella cibaria BLS13, and more specifically, to a Kimchi-derived new microorganism Weissella cibaria BLS13 which activates an inhibitory effect on avian influenza viruses, and to uses thereof. According to the present invention, the Weissella cibaria BLS13 or a culture medium thereof contains a substance having an anti-viral function, and thus can be used advantageously in feed additives, fermentation products, foods and medicine.

### TECHNICAL FIELD

The present invention relates to a new microorganism Weissella cibaria BLS13, and more particularly, to a Kimchi-derived new microorganism Weissella cibaria BLS13 which activates an inhibitory effect on avian influenza viruses, and to uses thereof.

### BACKGROUND ART

It has been observed in recent years that the human infections of highly pathogenic avian influenza (HPAI) are caused by avian H5N1, H7N7, and H9N2 viruses. The outbreak of a pandemic influenza may cause enormous personal damage including more than two million fatalities worldwide, social panic states, and economical paralysis, and thus the World Health Organization (WHO) demands for the preparation of supranational measures (release of Global Influenza Preparedness Plan, 2005).

Recently, the South Korean government confirmed the possibility of infection of mammals with avian influenza viruses, as well as suffered a drastic economic loss caused to the poultry industry and the social economy at the time of the occurrence of an avian influenza in 2003, 2006, and 2008 in Korea. In addition, there is caused a serious social problem associated with the outbreak of a pandemic influenza due to the occurrence of asymptomatic patients infected with the avian influenza in Korea.

Bacterial diseases caused by bacterial infection, including repiratory avian mycoplasmosis, colibacillosis, salmonellosis, and the like, as well as viral diseases also occur frequently in the Korean poultry industry. Thus, in the event of bacterial infection, the poultry may be further vulnerable to other diseases due to a decrease of immunity. Poultry farms use vaccines or antibiotics for the purpose of communicable disease control and hygiene management in order to prevent the damage of avian diseases. However, the inhibition of infection by inoculation of vaccines involves a problem of efficacy in the case where the type of an epidemic virus does not match to that of a vaccine. Thus, since the prevention of the avian diseases by the vaccine inoculation has a limitation, an effective treatment method or preventive measure against viruses has not been sufficiently prepared yet. Therefore, there is a need for a complex treatment and prevention agent which can reinforce an immune system of a poultry flock itself while exhibiting an inhibitory effect on pathogenic avian influenza viruses, and eliminate a stability problem.

It is known that a lactic acid bacteria can grow at a high rate in 20-40° C. irrespective of anaerobic or aerobic conditions, and lactic acid is produced to increase acidity and hydrogen peroxide or the like is produced during its culture such that the proliferation of pathogenic viruses and microorganisms is inhibited to prevent soil-borne diseases. In addition, the lactic acid bacteria produce various bioactive compounds, antiviral agent, antibacterial agent, and anticarcinogens to improve self-protection ability. Also, in case of livestock, stability of intestinal microbes, increase in feed efficiency, and increase in disease resistance are exhibited. However, in order to correctly use the lactic acid bacteria on an industrial basis, a procedure must be necessarily performed which definitely understand what properties suitable for the use purpose are from the scientific viewpoint rather than determination based on enumeration of affirmative effects of the lactic acid bacteria. The reason for this is that uniformity of products and reproducibility of effects can be ensured only through such a procedure.

Accordingly, the present inventors have made extensive efforts to isolate lactic acid bacteria which can be used to prevent avian influenza virus infection from 150 species of Kimchi-derived lactic acid bacteria and, as a result, have found that a new lactic acid bacteria Weissella cibaria BLS13 is selected and the growth of avian influenza viruses is inhibited by the ingredients of a culture medium of the lactic acid bacterial strain, thereby completing the present invention.

### DISCLOSURE OF INVENTION

### Technical Problem

An object of the present invention is to provide a new microorganism Weissella cibaria BLS13 [KCTC11516BP] having anti-viral function.

Another object of the present invention is to provide a feed composition, a pharmaceutical composition, a health supplement, a fermentation food, and a food additive, which contain Weissella cibaria BLS13 [KCTC11516BP] or a culture medium thereof.

Still another object of the present invention is to provide a method for preventing avian influenza virus infection in animals, which comprises orally administering a Weissella cibaria BLS13 [KCTC11516BP] or a culture medium thereof to animals.

### Technical Solution

In order to achieve the above object, the present provides a Weissella cibaria BLS13 [KCTC11516BP].

Also, the present invention provides an antiviral composition which contains a Weissella cibaria BLS13 [KCTC11516BP] or a culture medium thereof.

Also, the present invention provides a pharmaceutical composition for prevention of influenza infection, which contains a Weissella cibaria BLS13 [KCTC11516BP] or a culture medium thereof.

Also, the present invention provides a health supplement for prevention of influenza infection, which contains a Weissella cibaria BLS13 [KCTC11516BP] or a culture medium thereof.

Also, the present invention provides a fermentation food which contains a Weissella cibaria BLS13 [KCTC11516BP] or a culture medium thereof.

Also, the present invention provides a food additive composition which contains a Weissella cibaria BLS13 [KCTC11516BP] or a culture medium thereof.

Also, the present invention provides a feed additive for prevention of influenza infection, which contains a Weissella cibaria BLS13 [KCTC11516BP] or a culture medium thereof.

Also, the present invention provides a method for preventing avian influenza virus infection in animals, which comprises orally administering a Weissella cibaria BLS13 [KCTC11516BP] or a culture medium thereof to animals.

Other features and embodiments of the present invention will be more apparent from the following detailed descriptions and the appended claims.

### BEST MODE FOR CARRYING OUT THE INVENTION

Unless defined otherwise, all technical and scientific terms used herein have the same meaning as commonly understood by one of ordinary skill in the art to which the invention pertains. Generally, the nomenclature used herein and the experiment methods which will be described later are those well known and commonly employed in the art.

In one aspect, the present invention is directed to a new microorganism Weissella cibaria BLS13 [KCTC11516BP].

The Weissella cibaria BLS13 [KCTC11516BP] according to the present invention is a new lactic acid bacteria isolated from Kimchi. A molecular phylogenetic analysis based on 16S rDNA base sequences is performed on the new lactic acid bacteria, and as a result, the lactic acid bacteria was identified to be Weissella cibaria and was found to be a new microorganism. The Weissella cibaria BLS13 was deposited in KCTC (Korean Collection for Type Cultures) of KRIBB (Korea Research Institute of Bioscience and Biotechnology) on May 26, 2009. The Deposition No. is KCTC11516BP.

The Weissella cibaria is a gram positive bacillus belonging to the genus Weissella. The Weissella cibaria is a Kimchi-derived lactic acid bacteria which appears in the fermentation process of Kimchi as a product ripened by production of a lactic acid and fermented at low temperature after the mixing of radish, Chinese cabbage, and cucumber soaked in the salt water, or the soaked vegetables with seasonings such as red pepper, garlic, green opinion, ginger, salted seafood called “jeotgal”, and the like. The Weissella cibaria BLS13 according to the present invention is a microorganism which is isolated and identified newly.

The lactic acid bacteria according to the present invention exhibits a high inhibitory efficacy against avian influenza viruses to possess an excellent antiviral effect. In one Example of the present invention, it was found that lactic acid bacteria according to the present invention has an antiviral effect against an avian influenza virus H9N2.

In another aspect, the present invention is directed to an antiviral composition, a pharmaceutical composition for prevention of influenza infection, a health supplement for prevention of influenza infection, a fermentation food, a food additive composition, and a feed additive for prevention of influenza infection, each of which contains a Weissella cibaria BLS13 [KCTC11516BP] or a culture medium thereof.

As used herein, the term “culture medium” may refer to a culture stock solution comprising bacterial cells containing an active ingredient having an antiviral activity, a culture supernatant from which the bacterial cells had been removed, or a diluted solution of the culture medium.

In order to prepare an antiviral composition of the present invention, the Weissella cibaria BLS13 of the present invention can be cultured in large scale through a conventional liquid or solid culture technique. For formulations of the strain, the bacterial cells can be collected in a lyophilized, freeze-dried or hydrated state.

The Weissella cibaria BLS13 of the present invention exhibits an antiviral activity against avian influenza viruses.

The pharmaceutical composition according to the present invention refers to a microorganism formulation containing a Weissella cibaria BLS13 or a culture medium thereof. The pharmaceutical composition can be administered together with a pharmaceutically acceptable carrier base or nutrients by oral administration, parenteral administration, subcutaneous or intracavitary injection, or can be administered directly through a tube or catheter.

The pharmaceutical composition can be formulated in the form of tablets, capsules, implants, suppositories, powders, solutions, gels, strain suspension solutions, and the like.

The dose of the pharmaceutical composition can be determined by those skilled in the art and can vary depending on formulation method, administration type, and patient's age, body weight, sex, severity of disease, diet, administration frequency, administration route, excretion rate, and response sensitivity.

The fermentation food according to the present invention may include dairy products which can be refrigerated, frozen or stored for a long period of time, for example, Kimchi, milk, dry milk, yogurt, kefir, ice cream, milkshake, cheese, cream, curd, fermented milk, and fermentation food containing milk as well as soymilk, fermented cereals, patient's diets and infant's diets. Also, the fermentation food may be provided to animals as animal feed.

A substrate containing starch such as milk or cereals is preferably used for the culture of strains in order to culture the Weissella cibaria BLS13 of the present invention and produce it in the form of food or add it to food.

A nutritional composition prepared in the form of food or beverage may include one or more nutritional elements consisting of fats, proteins, carbohydrates, dietary fibers, minerals, and vitamins together with the new microorganism of the present invention. Among the nutritional elements, especially, the dietary fibers are generally known as a prebiotic substrate promoting cell division of lactic acid bacteria, and thus can be introduced in vivo to play a key role in the formation and maintenance of colonies in intestines. Therefore, the dietary fibers are contained in the composition of the present invention so that they can be administered after or simultaneously with ingestion of the composition.

The pharmaceutical composition, fermentation food, or food additive composition of the present invention may additionally contain a probiotic strain beneficial to the human body besides a pharmaceutically necessary component or a component for suitable for foods.

The amount of Weissella cibaria BLS13 contained in the pharmaceutical composition, fermentation food, or food additive composition may be about 10⁵ cfu/g to about 10¹¹ cfu/g, preferably about 10⁶ cfu/g to about 10¹° cfu/g, most preferably about 10⁷ cfu/g to about 10⁹ cfu/g. The strain is preferably administered in a viable state, or may be killed before ingestion or administered in an attenuated state. In addition, in the case where the strain is prepared using a culture supernatant, it may be additionally subjected to a sterilization process through a heat treatment process. The amount of the strain and dairy intake necessary to possess a minimum efficacy may vary depending on the bodily or health conditions of a patient who ingests the strain, but is preferably generally 10⁶ to 10¹² cfu/day, most preferably 10⁷ to 10¹⁰ cfu/day. The degree of antibacterial efficacy of the culture supernatant can be determined using a technique commonly used in the art and the dose of the culture supernatant can be determined accordingly.

In still another aspect, the present invention is directed to a method for preventing avian influenza virus infection in animals, which comprises orally administering a Weissella cibaria BLS13 [KCTC11516BP] or a culture medium thereof to animals.

In the present invention, the type of administrating the Weissella cibaria BLS13 [KCTC11516BP] or culture medium thereof to animals can be determined by those skilled in the art and may be in the form of feed additives, fermentation foods, freeze-dried formulations, diluted solutions of the culture medium of bacterial cells.

The oral administration of Weissella cibaria BLS13 [KCTC11516BP] or culture medium thereof can vary depending on factors such as administration type, to-be-administered animal's kind, age, body weight, severity of disease, excretion rate, and response sensitivity.

In the present invention, the orally administered animals may be mammals except human beings, preferably livestocks, most preferably poultry.

### EXAMPLES

Hereinafter, the present invention will be described in further detail with reference to examples. It will be obvious to those skilled in the art that these examples are illustrative purposes only and are not to be construed to limit the scope of the present invention.

It will be obvious to those skilled in the art that only an antiviral activity against H9N2 of Weissella cibaria BLS13 [KCTC11516BP] was confirmed in Examples below, but the Weissella cibaria BLS13 [KCTC11516BP possesses an inhibitory effect on even viruses activated through a mechanism similar to that of avian influenza viruses.

### Example 1

### Isolation of Lactic Acid Bacteria from Kimchi and Preparation of Antiviral Culture Medium

11 kinds of Kimchi prepared in a conventional Kimchi preparing method were selected, and the Kimchi was used as a Kimchi sample for isolation of lactic acid bacteria. The Kimchi sample was 10-fold diluted with 0.85 saline solution and the diluted solution was inoculated in an amount of 1 Ml each in an MRS agar medium (MRS agar, Difco.; 10 g of bacto peptone, 10 g of beef extract, 5 g of yeast extract, 20 g of glucose, 801 g of tween, 2 g of citric acid, 2 g of dibasic potassium phosphate, 5 g of sodium acetate, 0.1 g of manganese sulfate, 0.05 g of magnesium sulfate, and 15 g of agar per 1 l of distilled water), followed by streaking with a glass rod on the surface of a plate of the agar medium. Thereafter, the plate was cultured in a constant-temperature incubator of 30° C. for 48 hr to obtain colonies. Each of the obtained colonies was streaked on the MRS agar medium, was cultured at 30° C. for 48 hr, and thereby isolated a total of 150 colonies of lactic acid bacteria. 10 colonies determined to be dominant based on the type, color, and the like of the grown colonies were selected and secondarily cultured on an MRS medium. Thereafter, whether or not a bacteria which it is desired to isolate are sole strains was confirmed.

26 species of Kimchi-derived lactic acid bacteria selected as sole strains from the secondary culture were inoculated in 50m1 of MRS broth (Difco) and cultured stationarily at 30° C. for 24 hr. Then, the cultured medium was centrifigated at 6000 rpm for 15 min to remove bacterial cells therefrom, was neutralized to pH 6.5, and filtered by using a filter of 0.22 um pore size to prepare a culture medium of lactic acid bacteria.

### Example 2

### Isolation of Avian Influenza Viruses

A/chicken/Korea/LPM77/06 (H9N2)virus was used which had been isolated after inoculating feces collected from Korean native chicken in a traditional market situated at the six-way intersection, Cheongju-si, Choongcheong Buk-do, South Korea in an 11-day-old fertilized egg.

For the confirmation of influenza viruses, the extraction of viral RNA was performed by using RNeasy Mini Kit (Qiagen, Germany), and eight segments (i.e., HA, NA, M, NS, NP, PA, PB1, and PB2) of the extracted RNA were amplified in an RT-PCR method. As a result, it was confirmed by a gene analysis that the isolated virus was an H9N2 virus.

### Example 3

### Confirmation of Anti-Viral Activity of Kimchi-Derived Lactic Acid Bacteria Against Avian Influenza

An antiviral efficacy test was performed on the lactic acid bacteria selected in Example 1 in the following manner. A/chicken/Korea/LPM77/06 (H9N2) virus isolated in Example 2 was used as a low pathogenic avian influenza H9N2 serotype virus. An AIV LPM77 virus was cultured in a 9 to 10-day-old SPF embryonated egg and was proliferated so that it has at least a titer of 10⁸ EID₅₀/ml or more. Then, the proliferated virus was stored in a freezer maintained at −70° C. The culture medium of lactic acid bacteria was 10-fold diluted with distilled water and then maintained at 4° C. for use.

1.0 ml of 10-fold diluted viral solution (allantoic fluid) was mixed with 24 ml of distilled water, and a virus for reaction with the extract was prepared. The 10-fold diluted culture medium of lactic acid bacteria is introduced in an amount of 2.5 ml each into a test tube, and 2.5 ml of the prepared viral solution is introduced into the test tube and mixed with the 10-fold diluted culture medium in the test tube. Then, the resulting mixture (a total of 5 ml) was reacted for 45 min correctly while being well shaken each 10 min. In order to determine whether or not the virus is proliferated, when the reaction of the mixture is terminated, the reaction solution was diluted to 10⁻¹, 10⁻², 10⁻³, 10⁻⁴, 10⁻⁵, and 10⁻⁶, respectively, by using PBS, and 0.2 ml of reaction solution neutralized in four 10-day-old embryonated eggs per dilution factor was inoculated in an allantoic cavity. After inoculation, the inoculated reaction solution was cultured at 37° C. for 5 days, and an egg test was performed daily. Then, the embryonated eggs died within 24 hours after inoculation was regarded as an accidental death and excluded from the test performance. The inoculated eggs died within 5 days after 24 hours from inoculation was all stored at 4° C. An allantoic fluid was collected from all the embryonated eggs survived after 5 days of inoculation and the inoculated eggs stored at 4° C., respectively, and a hemagglutination test was performed on the collected allantoic fluid to determine whether the virus exists or not.

As a result, it could be found that the virus titer of a negative control is 10^{5.0}, whereas the virus titer of a strain after treatment of the culture medium of lactic acid bacteria is 10^{3.5}, which exhibits very high antiviral activity (see Table 1).

TABLE 1
Antiviral efficacy of culture medium of lactic acid bacteria

| | | Reduction amount of |
|---|---|---|
| | Viruses titer | viruses to control |
| | after treatment | group |
| | (EID₅₀) | (EID₅₀) |
| Culture medium of | 10^{3.5} | 10^{2.0} |
| lactic acid | | |
| bacteria Weissella | | |
| cibaria BLS13 | | |

| | |
|---|---|
| Control virus | 10^{5.5} |

### Example 4

### Identification of Strain of Weissella cibaria BLS13

For the identification of a strain of the lactic acid bacteria having an antiviral function confirmed in Example 3, a primer which can amplify a 16S rRNA encoding DNA portion was prepared based on 16S rDNA base sequences of lactic acid bacteria registered in a gene bank (GenBank, www.ncbi.nlm.nih.gov) so as to perform 16S rDNA base sequence analysis.

SEQ ID NO: 1:
5′-gac ggg tga gta aca cgt gg-3′
SEQ ID NO: 2:
5′-acg ggc ggt gtg tac-3′

Lysates of 10⁹ bacterial cells of lactic acid bacteria were used as templates and a pair of primers above was used to perform PCR to thereby obtain a 1308-bp DNA segment (SEQ ID NO: 3). The DNA segment was cloned in pGEM-Teasy vector (Promega Co.) to analyze the base sequences. As a result, it has been found that the selected lactic acid bacteria have a DNA homology of more than 99% with Weissella cibaria (Accession No FJ429987.1) or Weissella cibaria (Accession No FJ429985.1) etc. Based on the above results, a lactic acid strain having antiviral activity against avian influenza viruses is named “Weissella cibaria BLS13”, which was deposited in KCTC (Korean Collection for Type Cultures) of KRIBB (Korea Research Institute of Bioscience and Biotechnology) on May 26, 2009 (the Deposition No. is KCTC11516BP).

### INDUSTRIAL APPLICABILITY

As described above, according to the present invention, the Weissella cibaria BLS13 or a culture medium thereof contains a substance having an anti-viral function, and thus can be used advantageously in feed additives, fermentation products, foods, and medicines.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A Weissella cibaria BLS13 [KCTC11516BP].

2. An antiviral composition which contains a Weissella cibaria BLS13 [KCTC11516BP] or a culture medium thereof.

3. A pharmaceutical composition for prevention of influenza infection, which contains a Weissella cibaria BLS13 [KCTC11516BP] or a culture medium thereof.

4. A health supplement for prevention of influenza infection, which contains a Weissella cibaria BLS13 [KCTC11516BP] or a culture medium thereof.

5. A fermentation food which contains a Weissella cibaria BLS13 [KCTC11516BP] or a culture medium thereof.

6. A food additive composition which contains a Weissella cibaria BLS13 [KCTC11516BP] or a culture medium thereof.

7. A feed additive for prevention of influenza infection, which contains a Weissella cibaria BLS13 [KCTC11516BP] or a culture medium thereof.

8. A method for preventing avian influenza virus infection in animals, which comprises orally administering a Weissella cibaria BLS13 [KCTC11516BP] or a culture medium thereof to animals.
